(19) United States
(12) Patent Application Publication
(10) Pub. No.: US 2005/0239885 A1
(43) Pub. Date: Oct. 27, 2005

(54) Processes for the preparation of simvastatin

(75) Inventors: Narendra Shriram Joshi, Navi Mumbai (IN); Shekhar Bhaskar Bhirud, Navi Mumbai (IN); Kodali Eswara Rao, Navi Mumbai (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/112,893

(22) Filed: Apr. 22, 2005

Related U.S. Application Data
(60) Provisional application No. 60/564,420, filed on Apr. 22, 2004.

(51) Int. Cl. A61K 31/225; C07C 67/02

(52) U.S. Cl. 514/548; 560/256

(57) ABSTRACT

Improved processes for the preparation of 3-hydroxy-3-methylglutaryl-coenzyme A (HMG-CoA) inhibitors, e.g., simvastatin, and their intermediates are provided. In one embodiment, a process for the preparation of a carboxylic acid amine salt of formula I is provided 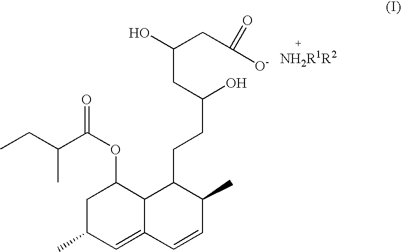
wherein R¹ and R² are as defined herein, the process comprising reacting lovastatin with an amine of formula III: 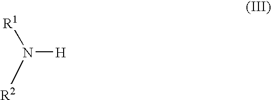
in an aqueous medium to provide the carboxylic acid amine salt of formula I. The process further includes the steps of lithiating the carboxylic acid amine salt of formula I to form the corresponding 2,2-dimethylbutyrate intermediate of formula IIa 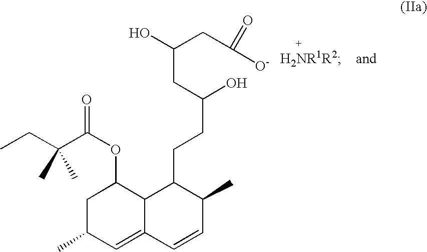 lactonizing the 2,2-dimethylbutyrate intermediate (IIa) to provide simvastatin and pharmaceutically acceptable salts thereof. Also provided is an improved process for lactonization of the intermediates herein using peptide coupling reagents.

### CROSS-REFERENCE TO RELATED APPLICATIONS

This application claims the benefit under 35 U.S.C. §119 to Provisional Application No. 60/564,420, filed Apr. 22, 2004 and entitled “PROCESS FOR THE PREPARATION OF SIMVASTATIN”, the contents of which are incorporated by reference herein.

### BACKGROUND OF THE INVENTION

1. Technical Field

The present invention generally relates to an improved process for the preparation of 3-hydroxy-3-methylglutaryl-coenzyme A inhibitors and their intermediates.

2. Description of the Related Art

The present invention is directed to an improved process for the preparation of 3-hydroxy-3-methylglutaryl-coenzyme A inhibitors and their intermediates, e.g., simvastatin (also known as butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8aβ]]). Simvastatin possesses the following structural formula: 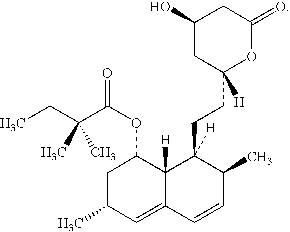
Generally, simvastatin is a synthetic lipid-lowering agent that acts as an inhibitor of 3-hydroxy-3-methylglutaryl-coenzyme A (HMG-CoA) reductase (HMG-CoA Reductase inhibitor). This enzyme catalyzes the conversion of HMG-CoA to mevalonate, an early and rate-limiting step in cholesterol biosynthesis. HMG-CoA reductase inhibitors are commonly referred to as “statins.” Statins are therapeutically effective drugs used for reducing low density lipoprotein (LDL) particle concentration in the blood stream of patients at risk for cardiovascular disease. Simvastatin is indicated for use for reducing elevated total cholesterol (total-C), low-density lipoprotein cholesterol (LDL-C), apolipoprotein B (Apo B), and high plasma triglycerides (TG), and to increase high-density lipoprotein cholesterol (HDL-C) in patients with primary hypercholesterolemia (heterozygous familial and nonfamilial) and mixed dyslipidemia (Fredrickson types Ia and IIb4); treating patients with hypertriglyceridemia (Fredrickson type IV hyperlipidemia); treating patients with primary dysbetalipoproteinemia (Fredrickson type III hyperlipidemia), and reducing total-C and LDL-C in patients with homozygous familial hypercholesterolemia as an adjunct to other lipid-lowering treatments (e.g., LDL apheresis). Simvastatin is sold under the trade name ZOCOR®.

HMG-CoA reductase inhibitors such as, for example, lovastatin, pravastatin, simvastatin, mevastatin, atorvastatin, cerivastatin, fluvastatin and analogs thereof, may be derived by fermentation or by chemical modification. Lovastatin, pravastatin and mevastatin are natural fermentation products which possess a 2-methylbutyrate side chain at the C₈ position of their hexahydronapthalene ring. Simvastatin is a semi-synthetic analog of lovastatin having a 2,2-dimethylbutyrate side chain at the C₈ position. The statins having a 2,2-dimethylbutyrate side chain, e.g. simvastatin, are synthesized because they are not naturally occurring compounds.

U.S. Pat. No. 4,444,784 (“the '784 patent”) discloses simvastatin. The '784 patent further discloses a process for preparing simvastatin by (1) de-esterification of the 2-methylbutylrate side chain; (2) protection of the 4-hydroxy of the pyranone ring; (3) re-esterification to form the desired 2,2-dimethylbutylrate; and (4) deprotection of the 4-hydroxy group. The process of the '784 patent is lengthy and provides poor overall yields.

One important aspect in the synthesis of simvastatin is the process of lactonization. Lactonization is a process where the hydroxyl acid loses one molecule of H₂O to form an intra-molecular ester, also known as a lactone. Lactonization is an equilibrium process characterized generally, in the case of statins, by equation I:

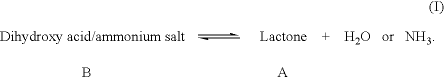

This reaction is generally catalyzed by an acid. The acidity necessary for this reaction is either inherent in the substrate itself or added by a lactonization agent, such as a strong acid. In order to obtain a high yield of the lactone, the equilibrium of the reaction must be shifted to the right hand side of the equation. The common way of shifting the equilibrium to the right is to remove a reaction product from the reaction mixture. One way of shifting the equilibrium to produce higher yields of lactone is by removing the H₂O produced by the reaction through azeotropic distillation. To perform the azeotropic distillation, either the free acid or the ammonium salt is heated in a suitable solvent, for example, toluene, butyl acetate, ethyl acetate, and cyclohexane, to a boiling point that forms an azeotrope mixture of solvent and water. The water, having a lower boiling point, is distilled off first and the reaction equilibrium is shifted to the right hand side towards the formation of lactone. The speed of water (and optionally ammonia) removal may be increased by passing a stream of inert gas through the reaction mixture. The ambient acidity of the statin acid is believed to be responsible for the lactonization reaction at these high temperatures.

U.S. Pat. No. 5,763,646 discloses a process for the preparation of simvastatin using lovastatin or a mevinolinic acid (the open ring form of lovastatin) salt as a starting material. The lovastatin or mevinolinic acid salt is reacted with an n-alkylamine or a cycloalkylamine of formula RNH₂, wherein R is C₃-C₆, e.g., cyclopropylamine or n-butyl amine, without requiring hydroxylprotection and subsequent deprotection. However, the use of the amine in opening the pyranone ring results in the formation of a lovastatin amide intermediate which may in turn undergo undesired side reaction due to the presence of its amide hydrogen atom. The undesired side reactions may further occur with the methylating agent thereby lowering the overall yield.

U.S. Pat. No. 6,603,022 discloses a process for the preparation of simvastatin using lovastatin as a starting material. The process involves (a) reacting lovastatin with a secondary amine such as diethylamine, pyrrolidine or piperidine to form an amide intermediate of the formula: 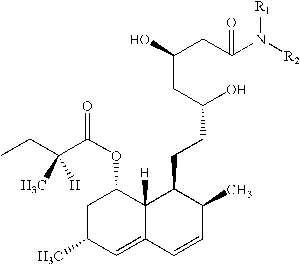
wherein R₁ and R₂ are each independently an alkyl, heteroalkyl, aryl or heteroaryl moiety, or R₁ and R₂, taken together, form a heterocyclic moiety having 5-8 atoms; wherein each of the foregoing alkyl and heteroalkyl moieties may be linear or branched, substituted or unsubstituted, cyclic or acyclic or saturated or unsaturated, and each of the foregoing heterocyclic, aryl and heteroaryl moieties may be substituted or unsubstituted; (b) methylating the C-8 butyrate side chain of the amide intermediate to form the corresponding 2,2-dimethylbutyrate intermediate, (c) hydrolyzing the 2,2-dimethylbutyrate intermediate to the corresponding free carboxylic acid and (d) effecting lactonization of the carboxylic acid intermediate to form simvastatin. This process is a four step process which includes the necessary step of hydrolyzing the 2,2-dimethylbutyrate intermediate to the corresponding free carboxylic acid prior to lactonization to form simvastatin.

Accordingly, there remains a need for improved processes for the preparation of simvastatin and pharmaceutically acceptable salts thereof that requires less steps thereby resulting in a more efficient process. There also remains a need for improved processes for the preparation of simvastatin and pharmaceutically acceptable salts thereof that eliminates and reduces the problems of the prior art on a commercial scale and in a convenient and cost efficient manner.

### SUMMARY OF THE INVENTION

One aspect of the present invention provides improved processes for the preparation of simvastatin and pharmaceutically acceptable salts thereof and its intermediates. The processes include at least the formation of a carboxylic acid amine salt in an aqueous medium, thus avoiding the use of an organic solvent. It also provides for lithiation of the carboxylic acid amine salt to provide the corresponding 2,2-dimethylbutyrate intermediate of the carboxylic acid amine salt and then lactonizing the 2,2-dimethylbutyrate intermediate of the carboxylic acid amine salt to provide simvastatin. The process of the present invention therefore avoids the additional steps required for amide formation and hydroxylprotection and deprotection as required by the prior art.

Accordingly, in a first embodiment of the present invention, a process for the preparation of a carboxylic acid amine salt of formula I is provided: 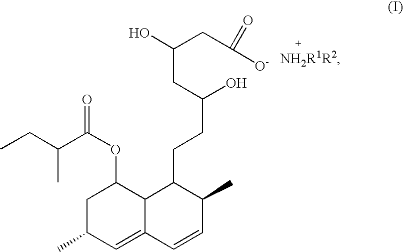
the process comprising reacting lovastatin of formula II: 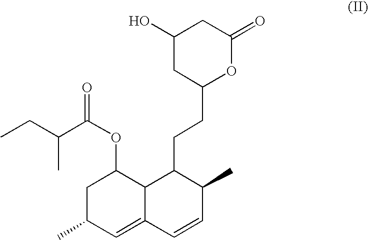
with an amine of formula III: 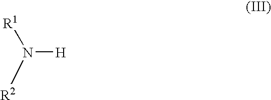
in an aqueous medium to provide the carboxylic acid amine salt of formula I.

In accordance with a second embodiment of the present invention, a process for the preparation of simvastatin and pharmaceutically acceptable salts thereof is provided comprising the steps of:
(a) providing a carboxylic acid amine salt of formula I: 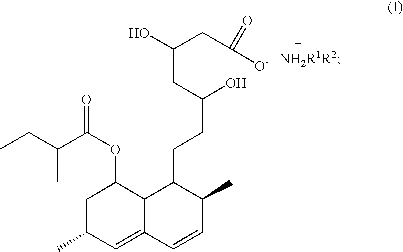
(b) lithiating the carboxylic acid amine salt (I) under suitable conditions to form the corresponding 2,2-dimethylbutyrate intermediate of formula IIa; 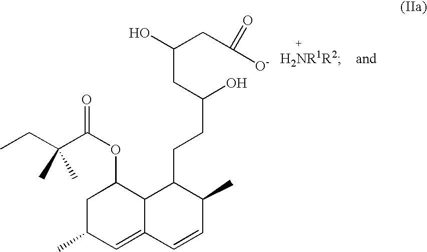
(c) lactonizing the 2,2-dimethylbutyrate intermediate (IIa) to provide simvastatin of formula IV

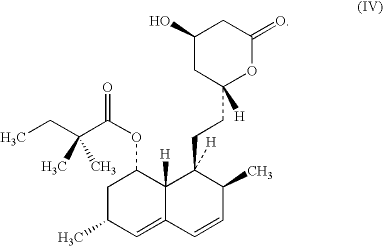

In accordance with a third embodiment of the present invention, a process for the preparation of simvastatin and pharmaceutically acceptable salts thereof is provided comprising the steps of:
(a) providing a carboxylic acid amine salt of formula I: 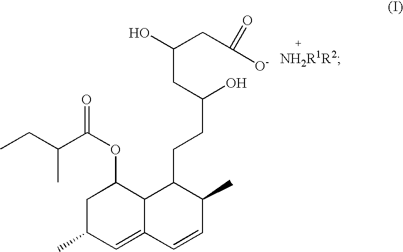
(b) lithiating the carboxylic acid amine salt (I) under suitable conditions to form the corresponding 2,2-dimethylbutyrate intermediate of formula IIa: 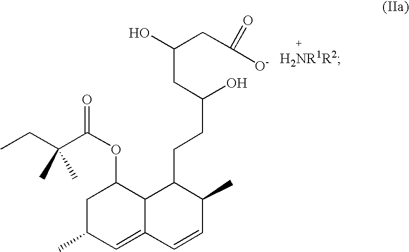
(c) converting the 2,2-dimethylbutyrate intermediate (IIa) to an ammonium salt of the 2,2-dimethylbutyrate intermediate of formula IIb: 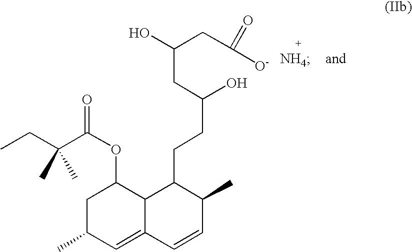
(d) lactonizing the ammonium salt of the 2,2-dimethylbutyrate intermediate (IIb) to provide simvastatin of formula IV:

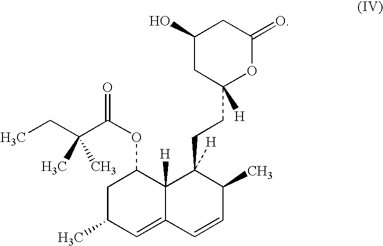

In accordance with a fourth embodiment of the present invention, a process for the preparation of simvastatin and pharmaceutically acceptable salts thereof is provided comprising the steps of:
(a) providing a carboxylic acid amine salt of formula I: 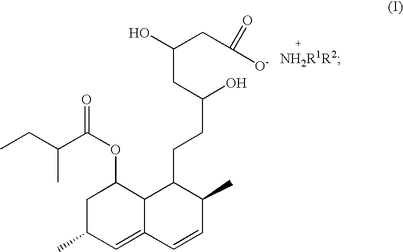
(b) lithiating the carboxylic acid amine salt (I) under suitable conditions to form the corresponding 2,2-dimethylbutyrate intermediate of formula IIa: 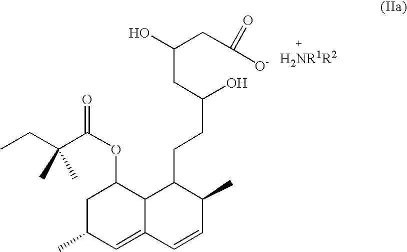
(c) converting the 2,2-dimethylbutyrate intermediate (Ia) to the corresponding free carboxylic acid; and
(d) lactonizing the free carboxylic acid intermediate to provide simvastatin of formula IV:

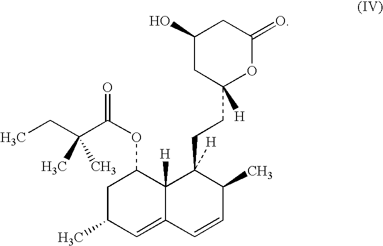

In accordance with a fifth embodiment of the present invention, a process for the lactonization of the corresponding free carboxylic acid of the 2,2-dimethylbutyrate intermediate (IIa) is provided comprising reacting the corresponding free carboxylic acid of the 2,2-dimethylbutyrate intermediate (IIa) with a peptide coupling reagent in the presence of an organic solvent.

In another aspect of the present invention, a process for the lactonization of the ammonium salt of the 2,2-dimethylbutyrate intermediate (IIa) is provided comprising lactonizing under reflux the ammonium salt of the 2,2-dimethylbutyrate intermediate (Ia) in a mixture of toluene and a polar aprotic solvent to provide simvastatin (IV) having an impurity content of less than about 0.15%.

Definitions

The term ‘alkyl’ as used herein means a straight or branched hydrocarbon chain radical containing carbon and hydrogen atoms of from 1 to about 8 carbon atoms, with no unsaturation, and which is attached to the rest of the molecule by a single bond, e.g., methyl, ethyl, n-propyl, 1-methylethyl (isopropyl), n-butyl, n-pentyl, 1,1-dimethylethyl (t-butyl), and the like.

The term “alkenyl” as used herein means an aliphatic hydrocarbon group containing a carbon-carbon double bond and which may be a straight or branched or branched chain having about 2 to about 10 carbon atoms, e.g., ethenyl, 1-propenyl, 2-propenyl(allyl), iso-propenyl, 2-methyl-1-propenyl, 1-butenyl, 2-butenyl and the like.

The term “alkynyl” as used herein means a straight or branched chain hydrocarbyl radicals having at least one carbon-carbon triple bond, and having in the range of about 2 up to about 12 carbon atoms (with radicals having in the range of about 2 up to about 10 carbon atoms being preferred) e.g., ethynyl, propynyl, butynyl and the like.

The term “alkoxy” as used herein means an alkyl group as defined above attached via oxygen linkage to the rest of the molecule, i.e., of the general formula —OR³, wherein R³ is an alkyl as defined above. Representative examples of those groups are —OCH₃, —OC₂H₅ and the like.

The term “cycloalkyl” as used herein means a non-aromatic mono or multicyclic ring system of about 3 to about 12 carbon atoms such as, for example, cyclopropyl, cyclobutyl, cyclopentyl; cyclohexyl, perhydronapththyl, adamantyl and norbornyl groups bridged cyclic group or sprirobicyclic groups e.g. sprio (4,4) non-2-yl and the like.

The term “cycloalkylalkyl” as used herein means a cyclic ring-containing radicals containing in the range of about 3 up to about 8 carbon atoms directly attached to the alkyl group which are then attached to the main structure at any carbon from alkyl group that results in the creation of a stable structure such as, for example, cyclopropylmethyl, cyclobutylethyl, cyclopentylethyl, and the like.

The term “cycloalkenyl” as used herein means a cyclic ring-containing radicals containing in the range of about 3 up to about 8 carbon atoms with at least one carbon-carbon double bond such as, for example, cyclopropenyl, cyclobutenyl, cyclopentenyl and the like.

The term “aryl” as used herein means an aromatic radicals having in the range of about 6 up to about 14 carbon atoms such as, for example, phenyl, naphthyl, tetrahydronapthyl, indanyl, biphenyl and the like.

The term “arylalkyl” as used herein means an aryl group as defined above directly bonded to an alkyl group as defined above. e.g., —CH₂C₆H₅, —C₂H₅C₆H₅ and the like.

The term “heterocyclic ring” as used herein means a stable 3- to about 15 membered ring radical, which consists of carbon atoms and from one to five heteroatoms selected from the group consisting of nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, isochromanyl and the like and mixtures thereof.

The term “heteroaryl” as used herein means a heterocyclic ring radical as defined above. The heteroaryl ring radical may be attached to the main structure at any heteroatom or carbon atom that results in the creation of a stable structure.

The heterocyclic ring radical may be attached to the main structure at any heteroatom or carbon atom that results in the creation of a stable structure.

The term “heteroarylalkyl” as used herein means a heteroaryl ring radical as defined above directly bonded to alkyl group. The heteroarylalkyl radical may be attached to the main structure at any carbon atom from alkyl group that results in the creation of a stable structure.

The term “heterocyclyl” as used herein means a heterocylic ring radical as defined above. The heterocylyl ring radical may be attached to the main structure at any heteroatom or carbon atom that results in the creation of a stable structure.

The term “heterocyclylalkyl” as used herein means a heterocylic ring radical as defined above directly bonded to alkyl group. The heterocyclylalkyl radical may be attached to the main structure at carbon atom in the alkyl group that results in the creation of a stable structure.

The substituents in the ‘substituted alkyl’, ‘substituted alkoxy’, ‘substituted alkenyl’, ‘substituted alkynyl’, ‘substituted cycloalkyl’, ‘substituted cycloalkylalkyl’, ‘substituted cyclocalkenyl’, ‘substituted arylalkyl’, ‘substituted aryl’, ‘substituted heterocyclic ring’, ‘substituted heteroaryl ring,’ ‘substituted heteroarylalkyl’, ‘substituted heterocyclylalkyl ring’, ‘substituted amino’, ‘substituted cyclic ring’ and ‘substituted carboxylic acid derivative’ may be the same or different with one or more selected from the group such as hydrogen, hydroxy, halogen, carboxyl, cyano, nitro, oxo (═O), thio(═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, substituted or unsubstituted heterocyclic ring, substituted or unsubstituted guanidine, —COORₓ, —C(O)Rₓ, —C(S)Rₓ, —C(O)NRₓR_{y}, —C(O)ONRₓR_{y}, —NRₓCONR_{y}R_{z}, —N(Rₓ)SOR_{y}, —N(Rₓ)SO₂R_{y}, —(═N—N(Rx)R_{y}), —NRₓC(O)OR_{y}, —NRₓR_{y}, —NRₓC(O)R_{y}—, —NRₓC(S)R_{y} —NRₓC(S)NR_{y}R_{z}, —SONRₓR_{y}—, —SO₂NRₓR_{y}—, —ORₓ, —ORₓC(O)NR_{y}R_{z}, —ORₓC(O)OR_{y}—, —OC(O)Rₓ, —OC(O)NRₓR_{y}, —RₓNR_{y}C(O)R_{z}, —RₓOR_{y}, —RₓC(O)OR_{y}, —RₓC(O)NR_{y}R_{z}, —RₓC(O)Rₓ, —RₓC(O)R_{y}, —SRₓ, —SORₓ, —SO₂Rₓ, —ONO₂, wherein Rₓ, R_{y} and R_{z} in each of the above groups can be the same or different and can be a hydrogen atom, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, ‘substituted heterocyclylalkyl ring’ substituted or unsubstituted heteroarylalkyl, substituted or unsubstituted heterocyclic ring.

### DETAILED DESCRIPTION OF THE INVENTION

The present invention is directed to the use of a carboxylic acid amine salt in the preparation of HMG-CoA reductase inhibitors, e.g., simvastatin, and their intermediates. One aspect of the present invention provides a process for the preparation of a carboxylic acid amine salt of formula I: 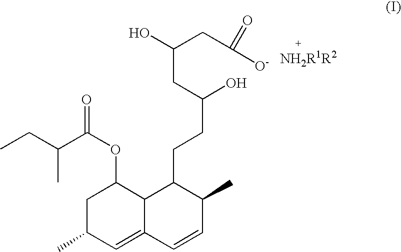
wherein R¹ and R² may be the same or different and may be hydrogen, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, with the proviso that only one of R¹ and R² can be hydrogen, or R¹ and R² together with the nitrogen atom to which they are bonded are joined together to form a heterocyclic ring, the process comprising reacting lovastatin of formula II: 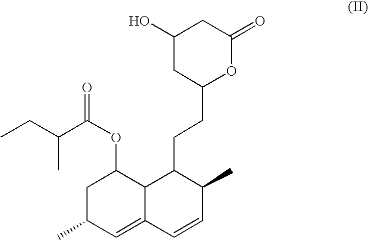
with an amine of formula III: 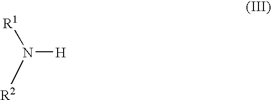
wherein R¹ and R² have the aforestated meanings; in an aqueous medium, e.g., water. In a preferred embodiment of the present invention, the amine is tert-butylamine, i.e., wherein R¹ is hydrogen and R² is tert-butyl, or n-butylamine.

The reaction of the compound of formula II with the amine can be carried out at a temperature of about 30° C. to about 100° C., preferably from about 40° C. to about 80° C. and more preferably from about 50° C. to about 60° C. The time period for the reaction to reach completion can range from about 1 hour to about 24 hours and preferably from about 3 hour to about 6 hours. Generally, the molar ratio of the compound of formula II to the amine of formula III can range from about 1:1.5 to about 1:3.5 and preferably from about 1:2 to about 1:2.2.

Another aspect of the present invention is a process for the preparation of simvastatin and pharmaceutically acceptable salts thereof including at least the steps of:
(a) providing a carboxylic acid amine salt of formula I;
(b) lithiating the carboxylic acid amine salt (I) under suitable conditions to form the corresponding 2,2-dimethylbutyrate intermediate of formula IIa: 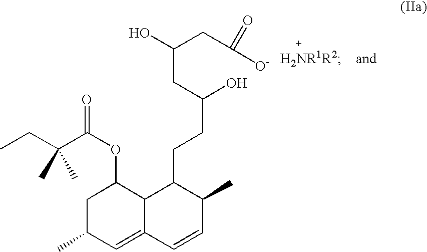
(c) lactonizing the 2,2-dimethylbutyrate intermediate (IIa) to provide simvastatin (IV).

In the first step of this process of the present invention, the carboxylic acid amine salt of formula (II) can be provided as described above or can be provided by methods known in the art. See, e.g., U.S. Pat. No. 6,583,295, the contents of which are incorporated by reference herein. For example, the carboxylic acid amine salt can be prepared by converting a sodium salt of lovastatin to the free acid of lovastatin by using phosphoric acid followed by reacting the free acid with a suitable amount of an amine to form an amine salt of lovastatin in ethyl acetate. The amine used in the above salt formation can be, for example, 1,2-dimethylpropylamine, 3-(2-aminoethylamino)-propylamine, N,N′-diisopropyl-ethylenediamine, N,N′-diethyl-ethylenediamine, N-methyl-1,3-propanediamine, N-methylethylenediamine, secondary-butylamine, tertiary-butylamine, tertiary-amylamine and secondary butylamine.

In step (b), the carboxylic acid amine salt (I) is lithiated with one or more lithiating agents under suitable conditions to form the corresponding 2,2-dimethylbutyrate intermediate (IIa). Suitable lithiating agents include, but are not limited to, methyl halides, methyl sulfonates and sulfates, methyl phosphates, methyl carbonates and the like and mixtures thereof. Representative examples of such lithiating agents include, but are not limited to, methyl iodide, methyl bromide, methyl p-toluenesulfonate, methanesulfonate and the like and mixtures thereof.

Generally, the lithiating step can be carried out in one or more organic solvents and in the presence of one or more base. Suitable solvents include, but are not limited to, tetrahydrofuran (THF), pyrrolidine, pyrrolidone, Et₂O, hexane and the like and mixtures thereof. The base for use herein can be organolithium compounds, alkali metal hydrides and the like and mixtures thereof. Suitable organolithium compounds include, but are not limited to, n-butyllithium, sec-butyllithium, tert-butyllithium, phenyllithium and the like and mixtures thereof. Suitable alkali metal hydrides include, but are not limited to, potassium hydride, sodium hydride and the like and mixtures thereof.

The carboxylic acid amine salt (I) can be lithiated with the one or more lithiating agents at a temperature ranging from about −10° C. to about −60° C. and preferably from about −25° C. to about −45° C. The amount of lithiating agent will ordinarily range from about 2.5 to about 8.0 equivalents and preferably about 3.0 to about 5.0 equivalents with respect to the equivalents of carboxylic acid amine salt (I).

The 2,2-dimethylbutyrate intermediate (IIa) is then subjected to lactonization by cyclizing the intermediate under suitable conditions to obtain the desired simvastatin of formula IV. The process of this embodiment is generally shown below in Scheme 1:

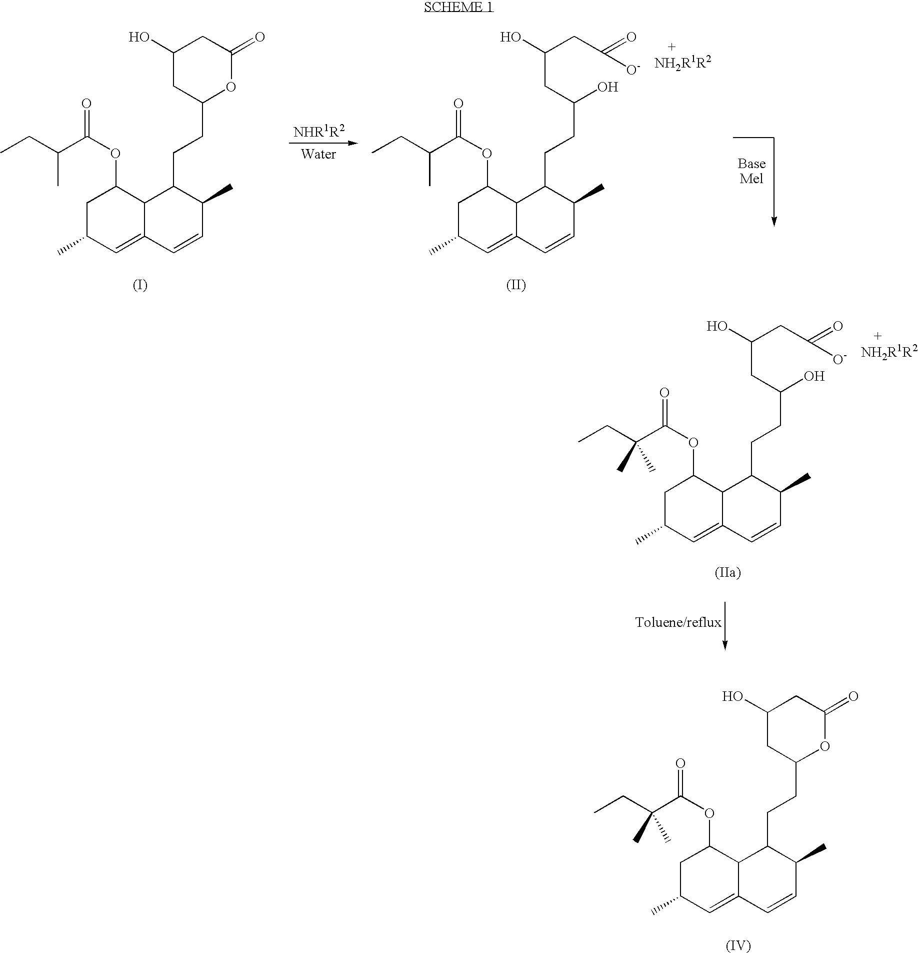

As shown in Scheme 1, the compound of formula II can be converted to a carboxylic acid amine salt (I) using an amine of formula III in an aqueous medium such as water at a temperature ranging from about 40° C. to about 80° C. The carboxylic acid amine salt (II) may then be lithiated using, e.g., a strong base, methyl iodide, and pyrrolidine in anhydrous THF, to provide the corresponding 2,2-dimethylbutyrate intermediate (IIa). The corresponding 2,2-dimethylbutyrate intermediate (IIa) may then be lactonized using conventional methods, e.g., azeotropic reflux with toluene, to provide simvastatin (IV). If desired, simvastatin can be converted to pharmaceutically acceptable salts thereof by known techniques.

Yet another aspect of the present invention is a process for the preparation of simvastatin and pharmaceutically acceptable salts thereof including at least the steps of:
(a) providing a carboxylic acid amine salt (I);
(b) lithiating the carboxylic acid amine salt amine salt (I) to provide the corresponding 2,2-dimethylbutyrate intermediate (IIa);
(c) converting the corresponding 2,2-dimethylbutyrate intermediate (IIa) to an ammonium salt of the 2,2-dimethylbutyrate intermediate (IIb); and,
(d) lactonizing the ammonium salt of the 2,2-dimethylbutyrate intermediate to provide simvastatin (IV). This process is generally shown below in Scheme 2:

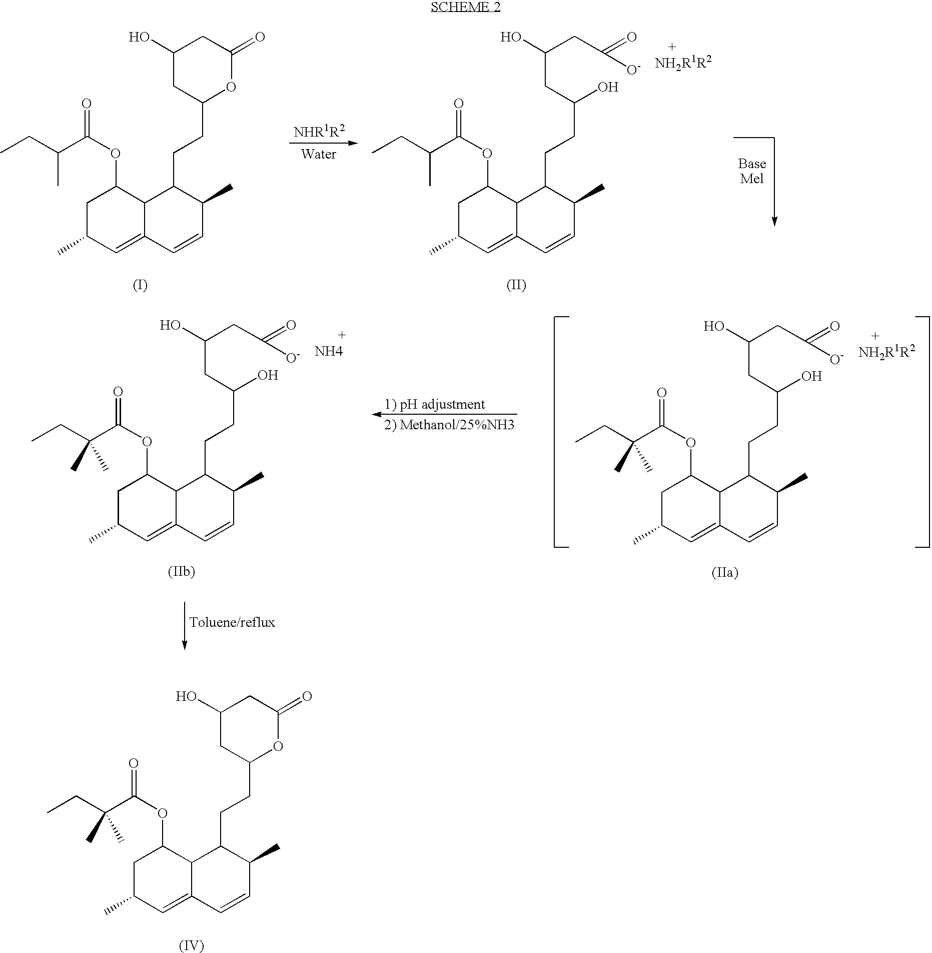

As shown in Scheme 2, lovastatin of formula (II) may be converted to a carboxylic acid amine salt (I) as described above. The carboxylic acid amine salt (I) may then be lithiated, e.g., in the presence of a strong base, methyl iodide (MeI) and a pyrrolidine in anhydrous tetrahydrofuran (THF), to provide the corresponding 2,2-dimethylbutyrate intermediate (IIa). The 2,2-dimethylbutyrate intermediate (IIa) may then be neutralized in situ to its free carboxylic acid using a suitable mineral acid, e.g., dilute hydrochloric acid (HCl). The free carboxylic acid may then be isolated using, for example, methanolic ammonia (about 25%), which forms an ammonium salt of the 2,2-dimethylbutyrate intermediate (IIb). The ammonium salt of the 2,2-dimethylbutyrate intermediate (IIb) may then be lactonized using conventional methods, e.g., azeotropic reflux with toluene, to yield simvastatin (IV).

Still yet another aspect of the present invention is a process for the preparation of simvastatin and pharmaceutically acceptable salts thereof including at least the steps of:
(a) providing a carboxylic acid amine salt (I);
(b) lithiating the carboxylic acid amine salt amine salt (I) to provide the corresponding 2,2-dimethylbutyrate intermediate (IIa);
(c) converting the corresponding 2,2-dimethylbutyrate intermediate (IIa) to its free carboxylic acid; and,
(d) lactonizing the free carboxylic acid of the 2,2-dimethylbutyrate intermediate to provide simvastatin. This process is generally shown below in Scheme 3:

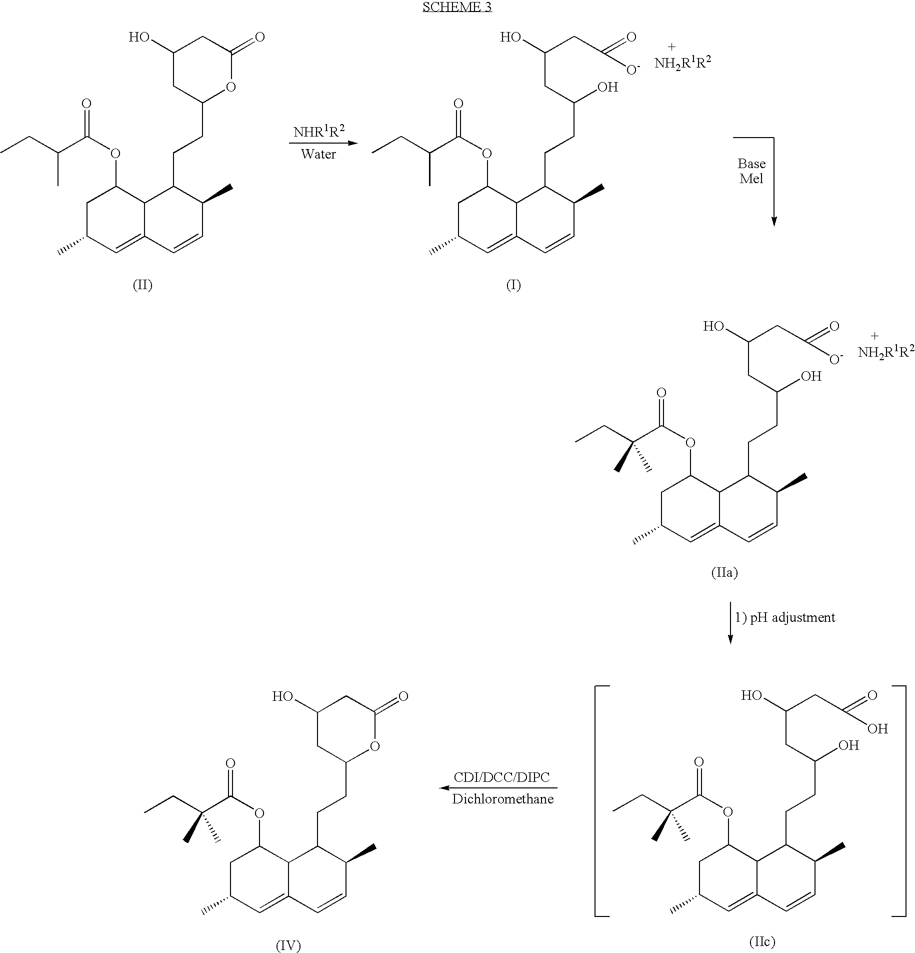

As shown in Scheme 3, lovastatin of formula (II) may be converted to a carboxylic acid amine salt (I) as described above. The carboxylic acid amine salt (I) may then be lithiated, e.g., in the presence of a strong base, methyl iodide and a pyrrolidine in anhydrous tetrahydrofuran (THF), to provide the corresponding 2,2-dimethylbutyrate intermediate (IIa). The 2,2-dimethylbutyrate intermediate (IIa) may then be neutralized in situ to its free carboxylic acid (IIc) using a suitable dilute acid mineral acid, e.g., dilute hydrochloric acid (HCl). The free carboxylic acid (IIc) may then be lactonized using, for example, a peptide coupling reagent in an organic solvent to form simvastatin (IV).

Suitable peptide coupling reagents for use herein include, but are not limited to, N,N¹ carbonyl diimidazole, dicyclohexyl carbodiimide, diisopropyl carbodiimide and the like and mixtures thereof. Suitable organic solvents for use herein include, but are not limited to, dichloromethane, chloroform, ethyl acetate and the like and mixtures thereof. Generally, the lactonization step using a peptide coupling reagents can be carried out at a temperature ranging from 0° C. to about 30° C. The amount of peptide coupling reagent will ordinarily range from about 1.0 to about 1.5 equivalents with respect to the free carboxylic acid.

Still yet another aspect of the present invention is a process for the preparation of simvastatin and pharmaceutically acceptable salts thereof including at least the steps of:
(a) providing a carboxylic acid amine salt (I);
(b) lithiating the carboxylic acid amine salt amine salt (I) to provide the corresponding 2,2-dimethylbutyrate intermediate (IIa);
(c) converting the corresponding 2,2-dimethylbutyrate intermediate (IIa) to an ammonium salt of the 2,2-dimethylbutyrate intermediate (IIb);
(d) neutralizing the ammonium salt of the 2,2-dimethylbutyrate intermediate (IIb) to provide the free carboxylic acid of the ammonium salt of the 2,2-dimethylbutyrate intermediate (IIc); and,
(e) lactonizing the free carboxylic acid to provide simvastatin (IV). This process is generally shown below in Scheme 4:

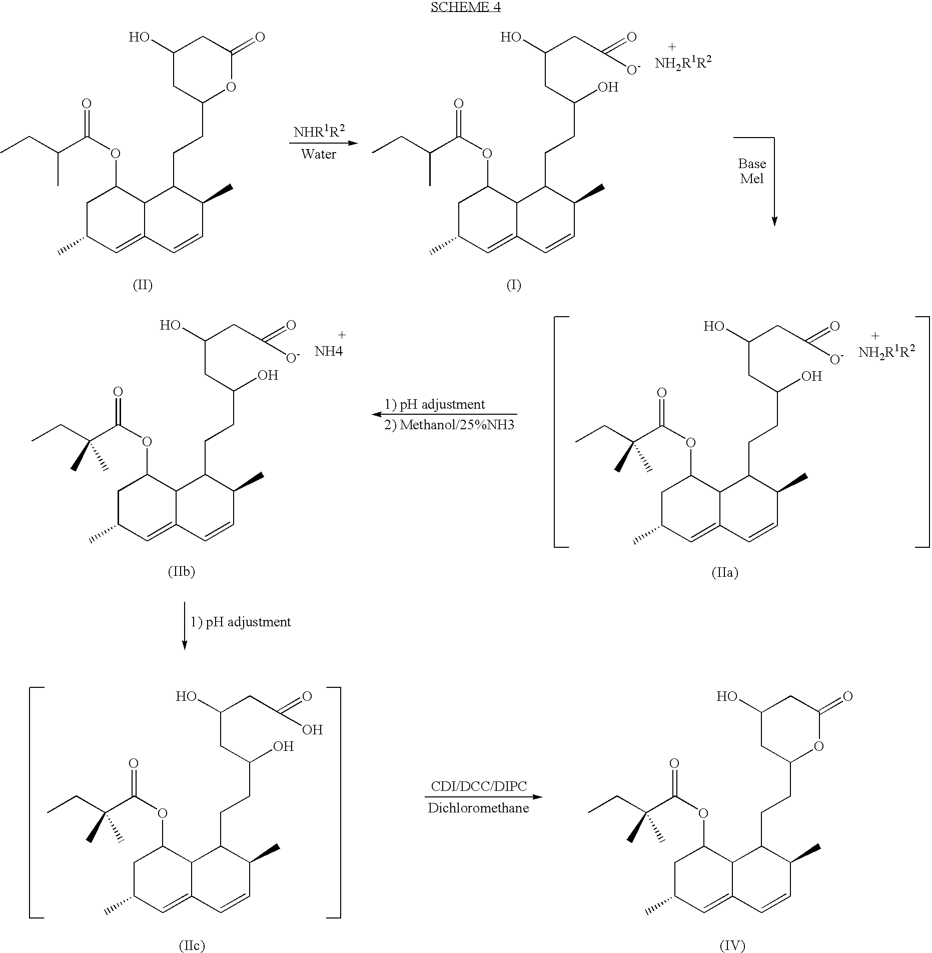

As shown in Scheme 4, lovastatin of formula (II) may be converted to a carboxylic acid amine salt (I) as described above. The carboxylic acid amine salt (I) may then be lithiated, e.g., in the presence of a strong base, methyl iodide and a pyrrolidine in anhydrous tetrahydrofuran (THF), to provide the corresponding 2,2-dimethylbutyrate intermediate (IIa). The 2,2-dimethylbutyrate intermediate (IIa) may then be neutralized in situ using a dilute acid, e.g., dilute hydrochloric acid, to yield a free carboxylic acid of the 2,2-dimethylbutyrate intermediate (IIb) and then isolated from the reaction mixture as an ammonium salt of the 2,2-dimethylbutyrate intermediate (IIc) by reaction with, for example, methanolic ammonia (about 25%). The ammonium salt of the 2,2-dimethylbutyrate intermediate (IIb) may then be neutralized using a dilute acid, e.g., a dilute hydrochloric acid, to provide a free carboxylic acid of the 2,2-dimethylbutyrate intermediate (IIc). The free carboxylic acid (5) may then be lactonized as described above, e.g., using a peptide coupling reagent in an organic solvent, to form simvastatin (IV).

In accordance with a fifth embodiment of the present invention, a process for the lactonization of the corresponding free carboxylic acid of the 2,2-dimethylbutyrate intermediate (IIa) is provided comprising reacting the corresponding free carboxylic acid of the 2,2-dimethylbutyrate intermediate (IIa) with a peptide coupling reagent in the presence of an organic solvent.

In another aspect of the present invention, a process for the lactonization of the ammonium salt of the 2,2-dimethylbutyrate intermediate (IIa) is provided comprising lactonizing under reflux the ammonium salt of the 2,2-dimethylbutyrate intermediate (IIa) in a mixture of toluene and polar aprotic solvent to provide simvastatin (IV) having an impurity content of less than about 0.15%. Polar aprotic solvent are DMF or DMAC or mixtures thereof.

The following examples are provided to enable one skilled in the art to practice the invention and are merely illustrative of the invention. The examples should not be read as limiting the scope of the invention as defined in the claims.

### EXAMPLE 1

### Preparation of Tert-butylamine Salt of Lovastatin

Into a 4-neck round-bottom flask, lovastatin (25 g) was added with water (250 ml) and tert-butylamine (13 ml). The reaction mixture was slowly heated to a temperature of about 50° C. over about 30 minutes. The reaction mixture was maintained at a temperature ranging between about 50° C. and about 55° C. for about 3 hours. After the completion of reaction as determined by TLC, the water was distilled out at a temperature below about 60° C., and the residue was stripped twice using acetone (2×50 ml). The residue was charged in acetone (200 ml) and stirred for about 1 hour at a temperature ranging from about 20° C. to about 25° C. The residue was filtered and washed with acetone. The material was dried at a temperature ranging from about 50° C. to about 55° C. This process yielded the tert-butylamine salt of lovastatin (27.2 g).

Yield of about 88%. HPLC purity greater than 99%.

IR (KBr) cm⁻¹:3513, 3326 (OH & NH str); 1715 (C═O str); 1537 (C—O str)

¹H NMR spectrum (CDCl₃) 300 MHz: δ6.0(d, J=9.3, 1H) δ5.8 (dd, J=6.3, 1H) δ5.51(br s, 1H) δ5.37(d, J=2.7, 1H) δ4.12(m, 1H) δ3.78 (m, 1H) δ2.42-1.45(m, 17H) δ1.34(s, 9H) 1.08(m, 6H), δ0.87(m, 6H).

Mass: m/z 496.4(M+).

### EXAMPLE 2

### Methylation of Lovastatin Tert-butylamine Salt

Into a 4-neck round-bottom flask, tetrahydrofuran (350 ml) and pyrrolidine (15 ml) were added together with the tert-butylamine salt of lovastatin of Example 1 under a nitrogen atmosphere. The reaction mass was cooled to a temperature of about −20° C. Next, N-butyl lithium (120 ml) was slowly added at a temperature ranging from about −20° C. to about −25° C. over about 45 minutes. The reaction mixture was maintained at this temperature for about an hour was and then cooled to a temperature of about −35° C. Tert-butylamine salt (20 g) was added in tetrahydrofuran (200 ml) to the reaction mixture, and stirred for about 1 hour at a temperature ranging from about −30° C. to about −35° C. Methyl iodide (5 ml) was added in a single lot at a temperature ranging from about −30° C. to about −35° C. and maintained for about 1 hour. Another lot of methyl iodide (5 ml) was added to the reaction mixture and maintained for about 3 hours at a temperature ranging from about −30° C. to about −35° C. The reaction mixture was warmed to a temperature of about −10° C. and quenched with water (200 ml). The mixture was stirred for 30 minutes and the layer of tetrahydrofuran and the aqueous layer were separated. The layer of tetrahydrofuran was washed with water (100 ml) and both the aqueous fractions were combined together. The resulting product was an aqueous solution of tert-butylamine salt of simvastatin.

### EXAMPLE 3

### Preparation of Ammonium Salt of Simvastatin

The aqueous solution of Example 2 was neutralized by adjusting the pH to about 4 using dilute hydrochloric acid, which yielded the free acid form of simvastatin. The free acid form of simvastatin was then extracted twice with ethyl acetate (300 ml). The organic layer was separated and dried over anhydrous sodium sulfate. Liquid ammonia (10 ml) in methanol (10 ml) was added and stirred for about 1 hour at a temperature ranging from about 20° C. to about 25° C. The reaction mixture was then cooled to a temperature of about 5° C. to obtain the desired ammonium salt of simvastatin. The product was filtered and washed with chilled ethyl acetate (20 ml) and then dried at a temperature of about 50° C.

Yield of about 79%. HPLC purity greater than 98.5%.

IR (KBr) cm⁻¹: 3449, 3276 (OH, NH str); 2961 (C—H str); 1717(C═Ostr) 1556(C—Ostr).

¹H NMR (CDCl₃) 300 MHz: δ5.95 (d, J=9.6, 1H) δ5.76 (dd, J=5.7, 1H) δ5.48 (br s, 1H) δ5.15 (d, J=2.4, 1H) δ3.81 (m, 1H) δ3.47 (m, 1H) δ2.50 (t, 1H) δ2.35-1.19 (m,16H) δ1.05 (m,9H) 0.8 (m,6H), δ0.87(m,6H).

Mass: m/z 437.4 (base ion).

### EXAMPLE 4

### Preparation of Simvastatin

The ammonium salt of simvastatin (10 g) of Example 3 was charged in toluene (500 ml) and heated to reflux. The water was separated azeotropically for about 7 hours. After completion of the reaction as determined by TLC, the mass was cooled to a temperature of about 60° C. Activated charcoal (2 g) was charged to the reaction mass and stirred for about 30 minutes. The reaction mass was filtered on celite and the toluene was distilled under vacuum at a temperature below about 50° C. to yield the lactone. Ethyl acetate (5 ml) and hexane (40 ml) were added to the lactone and heated to reflux at a temperature of about 65° C. The reaction was slowly cooled to a temperature of about 25° C. to about 30° C. and then cooled to a temperature of about 5° C. to obtain the desired simvastatin solid. The solid was filtered and washed with hexane (20 ml). The material was dried at a temperature of about 50° C.

Yield of about 91%. HPLC purity greater than 99%.

IR (KBr) cm⁻¹: 3555 (OH str.), 2965(═C—H str), 1712(C═Ostr), 1697(C═Ostr) 1262(C—O str). ¹H NMR (CDCl₃) 300 MHz: δ6.00(d, J=9.3, 1H) δ5.78 (dd, J=6, 1H) δ5.5(br t, 1H) δ5.37(q, 1H) δ4.6(m, 1H) δ4.38 (m, 1H) 62.78-1.33(m, 16H) δ1.13(m, 9H) 0.85(m, 6H) δ0.87(m, 6H).

Mass: m/z 419.4 (M+, base peak).

### EXAMPLE 5

### Preparation of Tert-Butylamine Salt of Lovastatin

Into a 4-neck round-bottom flask, lovastatin (25 g), water (250 ml) and tert-butylamine (13 ml) were added and slowly heated to a temperature of about 50° C. over 30 minutes. The reaction mixture was maintained at a temperature ranging from about 50° C. to about 55° C. for about 3 hours. After the completion of reaction as determined by TLC, the water was distilled out at a temperature below about 60° C. The residue was stripped twice using acetone (2×50 ml). The residue was charged in acetone (200 ml) and stirred for about 1 hour at a temperature ranging from about 20° C. to about 25° C. The reaction mass was then filtered and washed with acetone. The product was then dried at a temperature ranging from about 50° C. to about 55° C. yielding the tert-butylamine salt of lovastatin (27.2 g).

Yield of about 88%. HPLC purity greater than 99%.

IR (KBr) cm⁻¹: 3513, 3326 (OH & NH str.); 1715 (C═O str); 1537 (C—Ostr).

¹H NMR (CDCl₃) 300 MHz: δ6.0 (d, J=9.3, 1H) δ5.8 (dd, J=6.3, 1H) δ5.51 (br s, 1H) δ5.37 (d, J=2.7, 1H) δ4.12 (m, 1H) δ3.78 (m, 1H) δ2.42-1.45 (m, 17H) δ1.34 (s, 9H) 1.08 (m, 6H), δ0.87 (m, 6H).

Mass: m/z 496.4(M+).

### EXAMPLE 6

### Methylation of Lovastatin Tert-Butylamine Salt

Into a 4-neck round-bottom flask, tetrahydrofuran (350 ml) and pyrrolidone (15 ml) were added together with the tert-butylamine salt of lovastatin of Example 5 under a nitrogen atmosphere. The reaction mass was cooled to a temperature of about −20° C. Next, N-butyl lithium (120 ml) was slowly added to the reaction mass at a temperature ranging from about −20° C. to about −25° C. over about −45 minutes. The reaction mixture was maintained at that temperature for about an hour. The reaction mass was then cooled to a temperature of about −35° C. and tert-butylamine salt (20 g) was added in tetrahydrofuran (200 ml). The reaction mixture was stirred for about 1 hour at a temperature ranging from about −30° C. to about −35° C. Methyl iodide (5 ml) was added in single lot at a temperature ranging from about −30° C. to about −35° C. and maintained for about 1 hour. A second lot of methyl iodide (5 ml) was added and the reaction mixture was maintained for about 3 hrs. The reaction mixture was warmed to a temperature of about −10° C. and was quenched with water (200 ml). The mixture was stirred for about 30 minutes and the layers were separated. The tetrahydrofuran layer was washed with water (100 ml) and both the aqueous fractions were combined together, to provide an aqueous solution containing the tert-butylamine salt of simvastatin.

### EXAMPLE 7

### Preparation of Ammonium Salt of Simvastatin

The aqueous solution of Example 6 was neutralized by adjusting the pH to about 4 using dilute hydrochloric acid to yield the free acid form of simvastatin. The free acid was extracted twice with ethyl acetate (2×200 ml). The organic layer was separated and dried over anhydrous sodium sulfate. Liquid ammonia (10 ml) in methanol (10 ml) was added and stirred for about 1 hour at a temperature of about 20° C. to about 25° C. The reaction mass was then cooled to a temperature of about 5° C. to obtain the ammonium salt of simvastatin. The product was filtered and washed with chilled ethyl acetate (20 ml) and dried at a temperature of about 50° C.

Yield of about 79%. HPLC purity greater than 98.5%.

IR (KBr) cm⁻¹: 3449-3276 (OH & NH str.); 2961 (C—H str); 1717 (C═Ostr) 1556 (C-0 str). ¹H NMR (CDCl₃) 300 MHz: δ5.95(d, J=9.6, 1H) δ5.76 (dd, J=5.7, 1H) δ5.48 (br s, 1H) δ5.15 (d, J=2.4, 1H) δ3.81 (m, 1H) δ3.47 (m,1H) δ2.50 (t, 1H) δ2.35-1.19 (m,16H) δ1.05 (m,9H) 0.8 (m,6H), δ0.87 (m,6H).

Mass: m/z 437.4(base ion).

### EXAMPLE 8

### Lactonization of Simvastatin Free Acid

Into a 4-neck round-bottom flask, ethyl acetate (50 ml), water (25 ml) and the ammonium salt of simvastatin (5 g) of Example 7 were added together. The pH was adjusted to about 4.0 with dilute hydrochloric acid and stirred for about 30 minutes. The layers were separated. The aqueous layer was extracted with ethyl acetate (25 ml) and the organic layers were combined together and washed with brine. The organic layer was then dried over anhydrous sodium sulphate and distilled to yield an oily residue. The oily residue was stripped using dichloromethane (20 ml) to yield the simvastatin free acid (4.9 g). The free acid was dissolved in dichloromethane (50 ml) and cooled to a temperature of about 10° C. N,N¹ carbonyl diimidazole (2 g) was added in a single lot. The mixture was stirred for about 2 hours at a temperature ranging from about 10° C. to about 15° C. Upon completion of the reaction as determined by TLC, the dichloromethane was distilled out and the product was stripped with cyclohexane. The product was isolated in cyclohexane (50 ml) and then dried at a temperature of about 50° C.

Yield of about 85%. HPLC purity greater than 99%.

IR (KBr) cm⁻¹: 3548 (OH str.); 2967 (═C—H str); 1709 (C═Ostr) 1700 (C═Ostr) 1261(C—O str).

¹H NMR (CDCl₃) 300 MHz: Δ6.00 (d, J=9.3, 1H) δ5.78 (dd, J=6, 1H) δ5.5 (br t, 1H) δ5.37 (q, 1H) δ4.6 (m, 1H) δ4.38 (m, 1H) δ2.78-1.33 (m,16H) δ1.13 (m,9H) 0.85 (m,6H) δ0.87(m, 6H).

Mass: m/z 419.4(M+).

### EXAMPLE 9

### Lactonization of Simvastatin Free Acid

Into a 4-neck round bottom flask, ethyl acetate (250 ml), water (125 ml) and the ammonium salt of simvastatin (25 g) of Example 7 were added together. The pH was adjusted to about 4.0 with dilute hydrochloric acid and stirred for about 30 minutes. The layers were separated. The aqueous layer was extracted with ethyl acetate (100 ml) and the organic layers were combined together and washed with brine. The organic layers were then dried over anhydrous sodium sulphate and distilled to yield an oily residue. The oily residue was stripped using dichloromethane (50 ml) to yield the free acid of simvastatin (26.8 g). The free acid was dissolved in dichloromethane (250 ml) and cooled to a temperature of about 10° C. Dicyclohexyl carbodiimide (13 g) was added in single lot and the reaction mixture was stirred for about 2 hours at a temperature of about 10° C. to about 15° C. After the completion of the reaction as determined by TLC, the dichloromethane was distilled out and the product was striped with cyclohexane. The product was then isolated in cyclohexane (250 ml) at a temperature of about 50° C. This yielded the lactone form of simvastatin (21 g).

Yield of about 91%. HPLC purity greater than 99%.

IR (KBr) cm⁻¹: 3555 (OH str.); 2968 (═C—H str); 1710 (C═Ostr) 1697 (C═Ostr) 1260(C—O str).

The ¹H NMR spectrum (CDCl₃) δ6.00 (d, J=9.3, 1H) δ5.78 (dd, J=6, 1H) δ5.5 (br t, 1H) δ5.37 (q, 1H) δ4.6 (m, 1H) δ4.38 (m, 1H) δ2.78-1.33 (m, 16H) δ1.13 (m, 9H) 0.85 (m, 6H) δ0.87(m, 6H).

Mass: m/z 419.4 (M+).

### EXAMPLE 10

### Lactonization of Simvastatin Free Acid

Into a 4-neck round-bottom flask, ethyl acetate (50 ml), water (25 ml) and the ammonium salt of simvastatin (5 g) of Example 7 were added together. The pH was adjusted to about 4.0 with dilute hydrochloric acid and stirred for about 30 minutes. The layers were separated. The aqueous layer was extracted with ethyl acetate (25 ml), and the organic layers were combined together and washed with brine. The organic layers were then dried over anhydrous sodium sulphate and distilled to yield an oily residue. The oily residue was stripped using dichloromethane (20 ml) to yield the free acid of simvastatin (4.6 g). The free acid was dissolved in dichloromethane (50 ml) and cooled to a temperature of about 10° C. Diisopropyl carbodiimide (1.8 g) was added in a single lot and stirred for about 2 hours at a temperature ranging from about 10° C. to about 15° C. After completion of the reaction as determined by TLC, the dichloromethane was distilled out and the product was striped with cyclohexane. The product was then isolated cyclohexane (50 ml). The product was then dried at a temperature of about 50° C.

Yield of about 85%. HPLC purity greater than 99%.

IR (KBr) cm⁻¹: 3550 (OH str.); 2969 (═C—H str); 1710 (C═Ostr) 1698 (C═Ostr) 1267 (C—O str).

¹H NMR spectrum (CDCl₃) δ6.00 (d, J=9.3, 1H) δ5.78 (dd, J=6, 1H) δ5.5 (br t, 1H) δ5.37 (q, 1H) δ4.6 (m, 1H) δ4.38 (m, 1H) δ2.78-1.33 (m, 16H) δ1.13 (m, 9H) 0.85 (m, 6H) δ0.87(m, 6H).

Mass: m/z 419.4 (M+).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A process for the preparation of a carboxylic acid amine salt of formula I 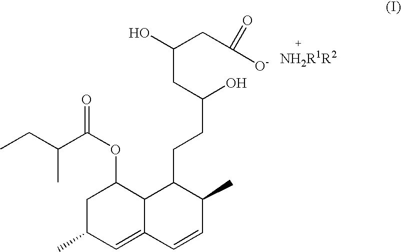
wherein R¹ and R² may be the same or different and can be hydrogen, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, with the proviso that only one of R¹ and R₂ can be hydrogen, or R¹ and R² together with the nitrogen atom to which they are bonded are joined together to form a heterocyclic ring; the process comprising reacting lovastatin of formula II: 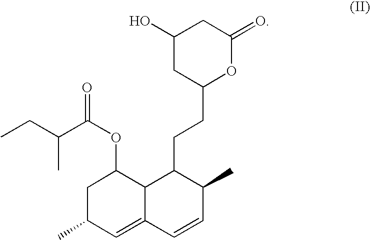
with an amine of formula III: 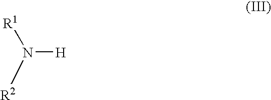
wherein R¹ and R² have the aforestated meanings, in an aqueous medium to provide the carboxylic acid amine salt of formula I.

2. The process of claim 1, wherein in the amine of formula III R¹ is hydrogen and R² is tert-butyl.

3. The process of claim 1, wherein the reaction is performed at a temperature of about 30° C. to about 100° C.

4. The process of claim 1, wherein the reaction is performed at a temperature of about 50° C. to about 60° C.

5. The process of claim 1 wherein the reaction is performed for a period of time of about 1 hour to about 24 hours.

6. The process of claim 1, wherein the aqueous medium comprises water.

7. The process of claim 1, further comprising lithiating the carboxylic acid amine salt (I) under suitable conditions to form the corresponding 2,2-dimethylbutyrate intermediate of formula IIa 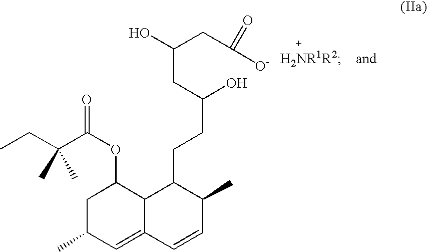
wherein R¹ and R² have the aforestated meanings.

8. The process of claim 7, wherein in the step of lithiating, the carboxylic acid amine salt (I) is reacted with one or more lithiating agents in the presence of a base in an organic solvent.

9. The process of claim 8, wherein the lithiating agent is selected from the group consisting of methyl halides, methyl sulfonates and sulfates, methyl phosphates, methyl carbonates and mixtures thereof.

10. The process of claim 8, wherein the lithiating agent is methyl iodide.

11. The process of claim 8, wherein the base is selected from the group consisting of organolithium compounds, alkali metal hydrides and mixtures thereof.

12. The process of claim 11, wherein the organolithium compounds are selected from the group consisting of n-butyllithium, sec-butyllithium, tert-butyllithium, phenyllithium and mixtures thereof.

13. The process of claim 11, wherein the alkali metal hydrides are selected from the group consisting of potassium hydride, sodium hydride and mixtures thereof.

14. The process of claim 8, wherein the organic solvent is selected from the group consisting of tetrahydrofuran (THF), pyrrolidine, pyrrolidone and mixtures thereof.

15. The process of claim 7, further comprising lactonizing the 2,2-dimethylbutyrate intermediate (IIa) to obtain simvastain of formula IV:

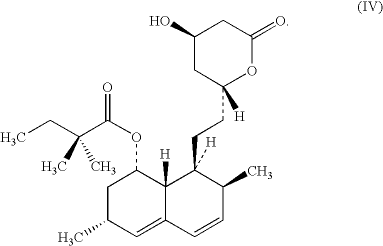

16. The process of claim 15, wherein the step of lactonizing comprises lactonizing the 2,2-dimethylbutyrate intermediate (IIa) under reflux in a solvent mixture comprising toluene to provide simvastatin (IV).

17. The process of claim 1, wherein the carboxylic acid amine salt (I) is thereafter converted to simvastatin or a pharmaceutically acceptable salt thereof.

18. The process of claim 1, further comprising
(b) lithiating the carboxylic acid amine salt (I) under suitable conditions to form the corresponding 2,2-dimethylbutyrate intermediate of formula IIa 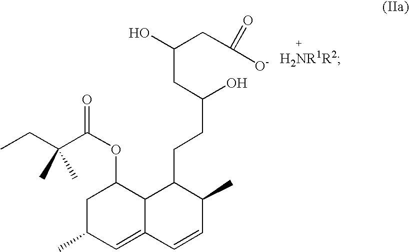
wherein R¹ and R² have the aforestated meanings;
(c) converting the 2,2-dimethylbutyrate intermediate (IIa) to an ammonium salt of the 2,2-dimethylbutyrate intermediate of formula (IIb): 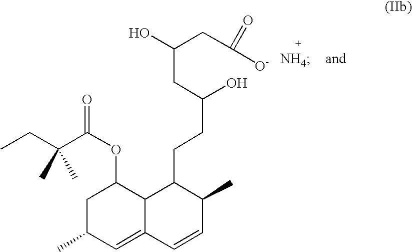
(d) lactonizing the ammonium salt to provide simvastatin of formula IV:

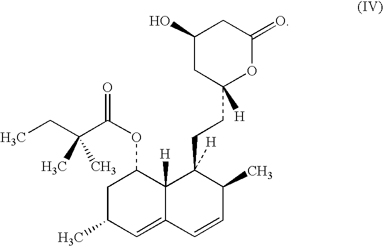

19. The process of claim 18, wherein the step of converting the 2,2-dimethylbutyrate intermediate (IIa) to an ammonium salt of the 2,2-dimethylbutyrate intermediate (IIb) comprises neutralizing in situ the 2,2-dimethylbutyrate intermediate (IIa) in the presence of a dilute acid to provide a free carboxylic acid of the 2,2-dimethylbutyrate intermediate and isolating from the reaction mixture the ammonium salt of the 2,2-dimethylbutyrate intermediate.

20. The process of claim 19, wherein the isolating step comprises reacting the free carboxylic acid of the 2,2-dimethylbutyrate intermediate with methanolic ammonia.

21. The process of claim 1, further comprising
(b) lithiating the carboxylic acid amine salt (I) under suitable conditions to form the corresponding 2,2-dimethylbutyrate intermediate of formula IIa 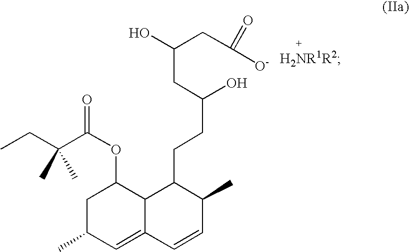
wherein R¹ and R² have the aforestated meanings;
(c) converting the 2,2-dimethylbutyrate intermediate (IIa) to an ammonium salt of the 2,2-dimethylbutyrate intermediate of formula (IIb): 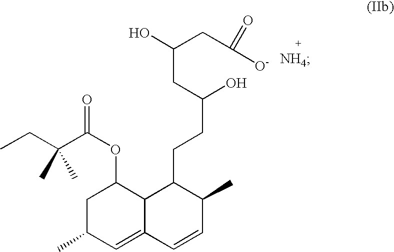
(d) neutralizing the ammonium salt of the 2,2-dimethylbutyrate intermediate (IIb) to provide a free carboxylic acid of the 2,2-dimethylbutyrate intermediate; and,
(e) lactonizing the free carboxylic acid of the 2,2-dimethylbutyrate intermediate to provide simvastatin of formula IV:

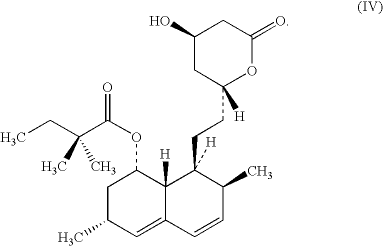

22. The process of claim 21, wherein in the step of lithiating, the carboxylic acid amine salt (I) is reacted with one or more lithiating agents in the presence of a base in an organic solvent.

23. The process of claim 21, wherein the step of lactonizing the free carboxylic acid of the 2,2-dimethylbutyrate intermediate to provide simvastatin comprises reacting the free carboxylic acid with a peptide coupling reagent in the presence of an organic solvent.

24. The process of claim 23, wherein the peptide coupling reagent is selected from the group consisting of N,N¹ carbonyl diimidazole, dicyclohexyl carbodiimide, diisopropyl carbodiimide and mixtures thereof.

25. The process of claim 23, wherein the organic solvent is selected from the group consisting of dichloromethane, ethylacetate, chloroform and mixtures thereof.

26. The process of claim 1, further comprising
(b) lithiating the carboxylic acid amine salt (I) under suitable conditions to form the corresponding 2,2-dimethylbutyrate intermediate of formula IIa 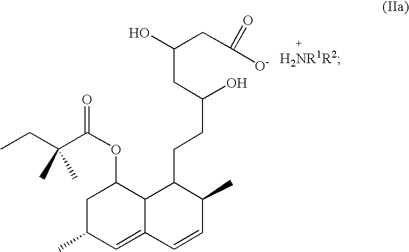
wherein R¹ and R² have the aforestated meanings;
(c) converting the 2,2-dimethylbutyrate intermediate (IIa) to a free carboxylic acid of the 2,2-dimethylbutyrate intermediate; and,
(d) lactonizing the free carboxylic acid of the 2,2-dimethylbutyrate intermediate to provide simvastatin of formula IV:

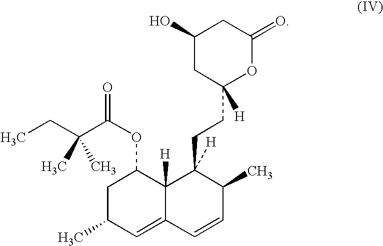

27. The process of claim 26, wherein in the step of lithiating, the carboxylic acid amine salt (I) is reacted with one or more lithiating agents in the presence of a base in an organic solvent.

28. The process of claim 26, wherein the step of converting the 2,2-dimethylbutyrate intermediate (IIa) to a free carboxylic acid of the 2,2-dimethylbutyrate intermediate comprises neutralizing the 2,2-dimethylbutyrate intermediate (IIa) in situ to its free carboxylic acid in the presence of a suitable dilute acid

29. The process of claim 28, wherein the suitable dilute acid is dilute hydrochloric acid.

30. The process of claim 26, wherein the step of lactonizing the free carboxylic acid of the 2,2-dimethylbutyrate intermediate to provide simvastatin comprises reacting the free carboxylic acid with a peptide coupling reagent in the presence of an organic solvent.

31. The process of claim 30, wherein the peptide coupling reagent is selected from the group consisting of N,N¹ carbonyl diimidazole, dicyclohexyl carbodiimide, diisopropyl carbodiimide and mixtures thereof.

32. The process of claim 30, wherein the organic solvent is selected from the group consisting of dichloromethane, ethylacetate, chloroform and mixtures thereof.

33. Simvastatin having a purity of greater than about 98% obtained from the process of claim 15.

34. Simvastatin having a purity of greater than about 98% obtained from the process of claim 16.

35. A process for the preparation of simvastatin and pharmaceutically acceptable salts thereof comprising the steps of:
(a) providing a carboxylic acid amine salt of formula I 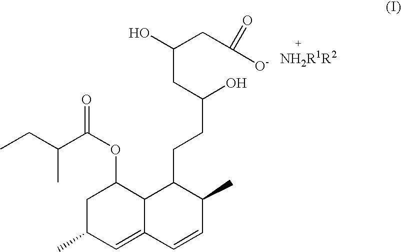
wherein R¹ and R² may be the same or different and can be hydrogen, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, with the proviso that only one of R¹ and R² can be hydrogen, or R¹ and R² together with the nitrogen atom to which they are bonded are joined together to form a heterocyclic ring;
(b) lithiating the carboxylic acid amine salt (I) under suitable conditions to form the corresponding 2,2-dimethylbutyrate intermediate of formula IIa 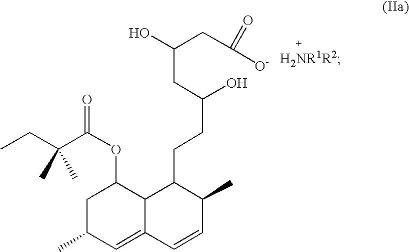
wherein R¹ and R² have the aforestated meanings; and
(c) lactonizing the 2,2-dimethylbutyrate intermediate (IIa) to provide simvastatin of formula IV:

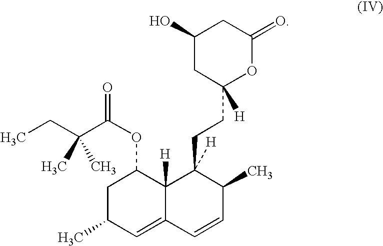

36. A process for the preparation of simvastatin and pharmaceutically acceptable salts thereof comprising the steps of:
(a) providing a carboxylic acid amine salt of formula I 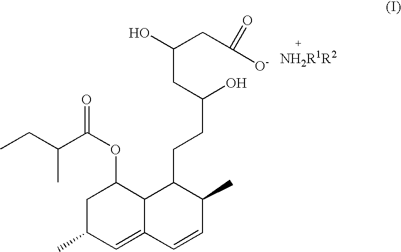
wherein R¹ and R² may be the same or different and can be hydrogen, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, with the proviso that only one of R¹ and R² can be hydrogen, or R¹ and R² together with the nitrogen atom to which they are bonded are joined together to form a heterocyclic ring;
(b) lithiating the carboxylic acid amine salt (I) under suitable conditions to form the corresponding 2,2-dimethylbutyrate intermediate of formula IIa 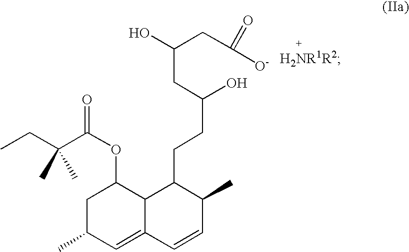
wherein R¹ and R² have the aforestated meanings; and
(c) converting the 2,2-dimethylbutyrate intermediate (IIa) to an ammonium salt of the 2,2-dimethylbutyrate intermediate of formula (IIb): 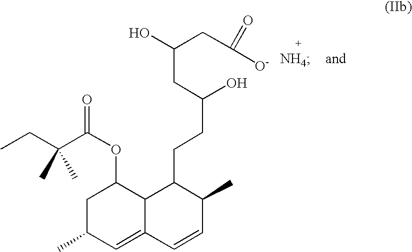
(d) lactonizing the ammonium salt of the 2,2-dimethylbutyrate intermediate to provide simvastatin of formula IV:

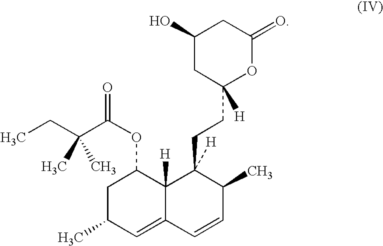

37. The process of claim 36, comprising the step of neutralizing the ammonium salt of the 2,2-dimethylbutyrate intermediate (IIb) prior to step (d) to provide a free carboxylic acid of the 2,2-dimethylbutyrate intermediate.

38. A process for the preparation of simvastatin and pharmaceutically acceptable salts thereof comprising the steps of:
(a) providing a carboxylic acid amine salt of formula I 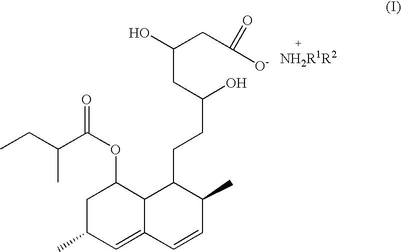
wherein R¹ and R² may be the same or different and can be hydrogen, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heterocyclic ring, substituted or unsubstituted heterocyclylalkyl, substituted or unsubstituted heteroarylalkyl, with the proviso that only one of R¹ and R² can be hydrogen, or R¹ and R² together with the nitrogen atom to which they are bonded are joined together to form a heterocyclic ring;
(b) lithiating the carboxylic acid amine salt (I) under suitable conditions to form the corresponding 2,2-dimethylbutyrate intermediate of formula IIa 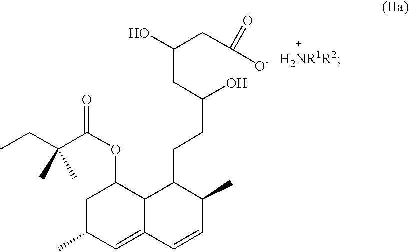
wherein R¹ and R² have the aforestated meanings; and
(c) converting the 2,2-dimethylbutyrate intermediate (IIa) to the corresponding free carboxylic acid; and
(d) lactonizing the free carboxylic acid intermediate to provide simvastatin of formula IV: [chemical structure]
